(19) United States
(12) Patent Application Publication
(10) Pub. No.: US 2018/0185312 A1
(43) Pub. Date: Jul. 5, 2018

(54) METHOD AND COMPOSITION FOR PREVENTING OR TREATING IMMUNE ALLERGIC AIRWAY DISEASE

(71) Applicant: Development Center for Biotechnology, NEW TAIPEI CITY (TW)

(72) Inventors: Jia-Ming Chang, New Taipei City (TW); I-Chun Ho, New Taipei City (TW)

(73) Assignee: Development Center for Biotechnology, NEW TAIPEI CITY (TW)

(21) Appl. No.: 15/394,856

(22) Filed: Dec. 30, 2016

(51) Int. Cl. A61K 31/198

(52) U.S. Cl. CPC A61K 31/198

(57) ABSTRACT

The present invention provides a composition for preventing or treating immune allergic airway disease, and uses of the composition in preparing drugs, wherein the composition comprises an effective amount of S-allyl-L-cysteine and a pharmaceutically acceptable carrier.

### BACKGROUND OF THE INVENTION

### Field of the Invention

The present invention relates to a method and a composition for preventing or treating immune allergic airway disease.

### Description of Prior Art

Allergic rhinitis, also known as nasal sensitivity, hay fever, pollinosis, pollen hypersensitivity or seasonal allergic rhinitis, is a collection of symptoms of rhinitis caused by airborne allergens affecting the immune system. Signs and symptoms include runny nose or stuffy nose, sneezing, red and itchy eyes and epiphora, and swelling around the eyes. The pathological causes for allergic rhinitis are as follows: 1, allergic reactions of the nasal cavity to changes in weather conditions, pollen, dust or specific allergens caused by congenital tracheal-bronchial abnormalities; 2, acquired allergic reactions, for example, allergies caused by long-term effect of allergens such as air pollution, drugs, pollen, etc., 3, neurological diseases, such as depression, can also induce allergic rhinitis. Usually after a patient is exposed or inhales allergens, IgE (immunoglobulin E) in the body will induce mast cells to release histamine, resulting in allergic reactions.

As to allergic rhinitis, there are currently drugs, desensitization therapy, surgical treatment, environmental control and other methods. Pharmacological therapy includes: antihistamine, vasoconstrictive agents, anti-inflammatory drugs, etc. For example: (a) antihistamine drugs: extremely effective in alleviating sneezing and runny nose, but some antihistamine drugs have side effects such as hypersomnia, and after long-term use, some antihistimine drugs become less effective or even ineffective in some patients; (b) anti-inflammatory drugs: there are mainly two categories, mast cell stabilizers (such as Intal Nasal Solution) and steroids (commonly known as American miracle elixir, such as Flixonase Aqueous Nasal Spray). A mast cell stabilizer only acts on the mast cells in the nasal mucosa to stabilize them and to reduce symptoms in the nose, it is mainly used as prevention, and often takes three to four weeks of continuous use in order to achieve best results; (c) steroid nasal spray; (d) antibiotics: when nasal mucosal bacterial infection is combined with sinusitis, then it is necessary to combine the use of antibiotics.

Non-pharmacological therapy includes: (a) surgical treatment: targeting at persistent nasal obstruction or combined with nasal septum deviation resulting from chronic inflammation, combined with turbinate hypertrophy. The surgical methods are turbinate reduction or turbinectomy, nasal mucosal cautery, sinus surgery, etc. (b) environmental control: avoiding contact with surrounding allergens. (c) desensitization therapy: administering small amounts of purified allergens into the skin of a patient's forearm for stimulation of protective antibody production by the patient's own immune system so that the patient is immune from allergen stimulation. However, not all therapies are effective, and it is relatively time consuming, requiring two to three years of regular injections.

Aged garlic extract, AGE, is an odorless product extracted from fresh garlic at room temperature after a long period of time. AGE is highly bioavailable and has high biological activity in humans and animals. The compositional changes of AGE are shown in the following table. S-allyl-L-Cysteine (SAC) is the main component of AGE. Currently, animal experiments and in vitro tests show that AGE has the effect of anti-inflammation, anti-oxidation, lowering blood pressure, antithrombosis, inhibiting platelet aggregation, preventing and treating cardiovascular disease, regulating blood sugar, improving neurological-related diseases, improving immunotoxic blood toxicity, and impaired burn healing, etc. However, up until now no research studies have reported the application of SAC to allergic rhinitis.

### SUMMARY OF THE INVENTION

The present invention provides a method for using a composition to prepare a drug for preventing or treating immune allergic airway disease, wherein the composition comprises an effective amount of S-allyl-L-Cysteine (SAC), and a pharmaceutically acceptable carrier.

### BRIEF DESCRIPTION OF THE DRAWINGS

FIG. 1 shows experimental design for animal models.

FIG. 2 shows the frequency of two monitored symptoms in the experiment: sneezing and nasal rubbing.

FIG. 3 shows the levels of OVA-specific immunoglobulin E.

FIG. 4 shows changes in cytokines in nasal irrigation solution.

FIG. 5 shows the nasal histopathological sections stained with PAS staining.

### DETAILED DESCRIPTION OF THE INVENTION

Unless otherwise defined herein, the scientific and technical term used in the present invention should have the meaning commonly understood by those skilled in the art. The meaning and scope of these terms should be clear; however, in any potential ambiguity, the definitions provided herein are preferred over any dictionary or external definition.

As utilized in accordance with the present disclosure, the following terms, unless otherwise indicated, shall be understood to have the following meanings.

The term “immune allergic airway disease” as used herein refers to an immune allergic disease affecting airways including the nasal passages, bronchi, and lungs. It ranges from acute infections (such as pneumonia and bronchitis) to chronic illnesses (such as asthma and chronic obstructive pulmonary disease).

The term “preventing” as used herein refers to delaying the onset of symptoms or reducing occurrences of diseases of a subject suffering from diseases.

The term “treating” as used herein refers to alleviating or improving the symptoms of a subject suffering from diseases.

The term “subject” as used herein refers to an animal, especially a mammal. In a preferred embodiment, the term “subject” refers to a “human.”

The term “an effective amount” as used herein refers to the amount of the active ingredient alone or in combination with other pharmaceutical compositions which shows to be effective in preventing or treating airway diseases.

For example, when being administered into a mouse, an effective amount of SAC is at least approximately 25 mg/kg; more preferably, an effective amount of SAC is approximately from 25 mg/kg to 100 mg/kg. When being administered into a person, an effective amount of SAC is at least approximately 2.75 mg/kg; more preferably, an effective amount is approximately from 2.75 mg/kg to 11 mg/kg. The conversion of an effective amount from animal to animal can be easily calculated by those skilled in the art based on known knowledge. For example, when an effective amount per kg for animal A is known and an effective amount per kg for animal B is to be calculated, look up table 1 first to find the conversion factor (W), then calculate as follows:

An effective amount for animal B (mg/kg)=W×an effective amount for animal A (mg/kg)

For example, in one of the examples of this disclosure, an effective amount of SAC being administered into a mouse was 25 mg/kg, which needs to be converted into an effective amount for a human. Animal A is a mouse and animal B is a human, the crossing point which is the conversion factor W is 0.11, therefore an effective amount for a human is 0.11×25 mg/kg=2.75 mg/kg.

TABLE 1
Animal and human body weight weight conversion factor per kilogram table

| | | Group A animal or adult | | | | | | |
|---|---|---|---|---|---|---|---|---|
| | | Mouse | Rat | Guinea pig | Rabbit | Cat | Dog | Adult |
| Conversion factor W | | 0.02 kg | 0.2 kg | 0.4 kg | 1.5 kg | 2 kg | 12 kg | 60 kg |
| Group B | Mouse 20 kg | 1.0 | 1.6 | 1.6 | 2.7 | 3.2 | 4.8 | 9.01 |
| animal | Rat 0.2 kg | 0.7 | 1.0 | 1.14 | 1.88 | 2.3 | 3.6 | 6.25 |
| or adult | Guinea Pig 0.4 kg | 0.61 | 0.87 | 1.0 | 1.65 | 2.05 | 3.0 | 5.55 |
| | Rabbit 1.5 kg | 0.37 | 0.52 | 0.6 | 1.0 | 1.23 | 1.76 | 2.30 |
| | Cat 2.0 kg | 0.30 | 0.42 | 0.48 | 0.81 | 1.0 | 1.44 | 2.70 |
| | Dog 12 kg | 0.21 | 0.28 | 0.34 | 0.56 | .068 | 1.0 | 1.88 |
| | Adult 60 kg | 0.11 | 0.16 | 0.18 | 0.304 | 0.371 | 0.531 | 1.0 |

(Source) http://www.39kf.com/cooperate/book/05/18/2006-01-13-163604.shtml)

The present application provides a method for preventing or treating immune allergic airway disease in a subject in need thereof, comprising administering a composition comprising an effective amount of S-allyl-L-cysteine and a pharmaceutically acceptable carrier.

In a preferred embodiment, immunoglobulin E is as immune indicator for the immune allergic airway disease.

In a preferred embodiment, the immune allergic airway disease is selected from allergic rhinitis.

In a preferred embodiment, the allergic rhinitis comprises symptoms of sneezing and nasal rubbing.

In a preferred embodiment, the effective amount of S-allyl-L-cysteine for mice is at least about 25 mg/kg.

In another preferred embodiment, the effective amount of S-allyl-L-cysteine for human is at least about 2.75 mg/kg.

In a preferred embodiment, the pharmaceutically acceptable carrier comprises a diluent, an excipient, or an acceptance agent.

The present invention provides a method for using a composition to prepare a drug for preventing or treating immune allergic airway disease, wherein the composition comprises an effective amount of S-allyl-L-cysteine (SAC), and a pharmaceutically acceptable carrier.

In a preferred embodiment, immunoglobulin E is an immune indicator for the immune allergic airway disease.

In another preferred embodiment, the immune allergic airway disease is selected from bronchitis, obstructive bronchitis, spastic bronchitis, allergic bronchitis, asthma, chronic obstructive pulmonary disease (COPD), and allergic, seasonal or perennial rhinitis. More preferably, the airway disease is selected from asthma, chronic obstructive pulmonary disease (COPD) or allergic rhinitis.

In another preferred embodiment, symptoms of the allergic rhinitis include sneezing and nasal rubbing.

In a preferred embodiment, an effective amount of the S-allyl-L-cysteine for a mouse is at least approximately 25 mg/kg.

In another preferred embodiment, an effective amount of the S-allyl-L-cysteine for a human is at least approximately 2.75 mg/kg.

In another preferred embodiment, the aforementioned composition further comprises one or a plurality of other drugs for preventing or treating airway disease.

In a preferred embodiment, the pharmaceutically acceptable carrier comprises a diluent, an excipient, an acceptance agent, or the like.

The present invention further provides a use of the aforementioned composition for preparing a food product. In a preferred embodiment, the food product is selected from the group consisting of food, beverages, health food, drugs, and animal feed.

### Examples

Drug Efficacy Evaluation for Allergic Rhinitis in Animals

Materials and Methods:

Experimental Animals

1. Species: Mice.

2. Strain: BALB/c.

3. Source: BioLASCO Taiwan Co., Ltd.

4. Age: 6 weeks.

5. Animal quarantine: Animals had been quarantined for one week and examined by a veterinarian in the animal house before entering the feeding room.

6. Body weight: 18-20 g.

7. Numbering markers: numbered ear tags were used for numbering/marking the animals, each cage was provided with an identification card stating the cage number, group, sex, and animal number.

8. Number of animals: The animals were divided into 5 groups, 4 to 5 animals per group, a total of 24 animals.

9. Feeding environment: The temperature of the feeding room was set at 21±2° C., humidity at 30-70%, and 12-hour light, 12-hour dark cycles. Feed (Purina Certified Rodent Chow) and water were provided ad libitum.

The number of animal experiments and the methods of the animal house were reviewed by the Institutional Animal Care and Use Committee (IACUC) of the Development Center for Biotechnology, the permit number was 2015-R501-041.

Reagents and Instruments

1. Albumin from chicken egg white (Ovalbumin) (Sigma)

2. Imject™ Alum Adjuvant (Thermo Fisher Scientific Inc.)

3. Micropipettes, micropipette tips, injection syringes

4. 10% formalin solution (J. T. Baker)

Animal Models

| | | | | Inoculation | |
|---|---|---|---|---|---|
| | | Animal | | Administration | Pathology |
| Group | | Distribution | Species | route | Symptoms |
| 1 | Control | 5♂ | BALB/ | Oral | Observation of |
| | group | | c | administration | clinical symptoms: |
| 2 | Disease | 5♂ | | (p.o.) | sneezing and nasal |
| | group | | | (twice per day) | rubbing |
| 3 | SAC | 4♂ | | | |
| | 25 mpk | | | | |
| 4 | SAC | 5♂ | | | |
| | 50 mpk | | | | |
| 5 | SAC | 5♂ | | | |
| | 100 mpk | | | | |
| | Total | n = 24 | | | |

mpk: milligrams per kilogram

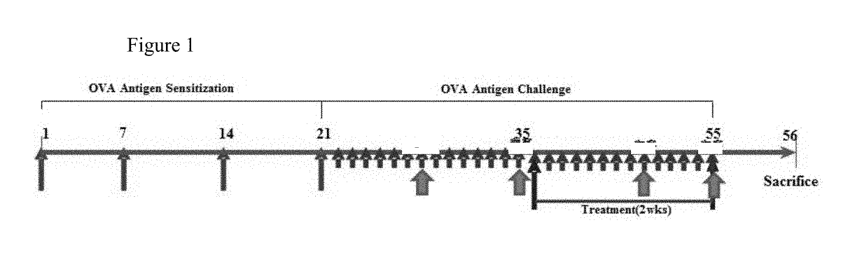

Experimental design for animal models as shown in FIG. 1.

Indicators

1. The frequency of sneezing and nasal rubbing of the mice was observed once per week.

2. Blood was collected from the heart of all experimental animals before they were sacrificed. Serum was obtained after the blood was centrifuged at 13000 rpm for 10 minutes at 4° C., then stored at −80° C. In addition, complete blood count (CBC) of the whole blood (including heparin-1:20) was determined.

3. All animals were sacrificed at the end of the 4th week after being given the tested material, their nasal cavities were rinsed 3 times with 1 ml of physiological saline solution, then nasal irrigation solution was collected, centrifuged at 1000 rpm for 5 minutes at 4° C. to collect the supernatant solution, then store at −80° C. for preservation.

4. After the nasal irrigation solution was collected, the entire nasal cavities of the mice were excised, stored in 10% formalin solution for preservation and used for histopathological sections.

5. Sacrifice collected spleen grind and frozen cells

6. Observation of histopathological sections: HE and AB/PAS staining

Results and Discussion

The frequencies of sneezing and nasal rubbing, the two monitored symptoms during the test, are shown as FIG. 1. In comparison to the disease group, after SAC treatment, the frequencies of sneezing and nasal rubbing tended to decline.

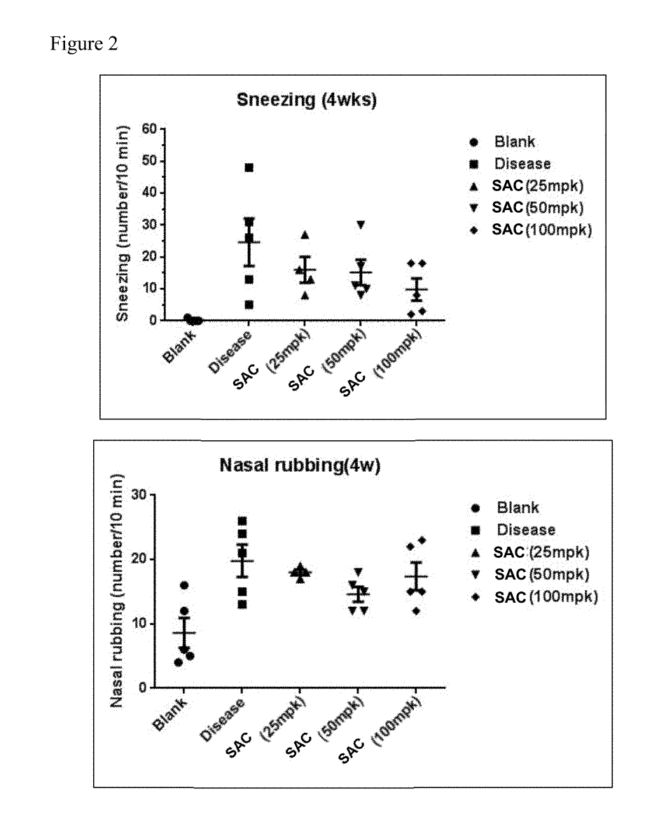

The OVA-IgE levels in the nasal irrigation solution of the disease group increased in comparison to those in the control group, when different amount of tested materials were given to all groups, the OVA-IgE level in the nasal irrigation solution showed dose-dependency (FIG. 2).

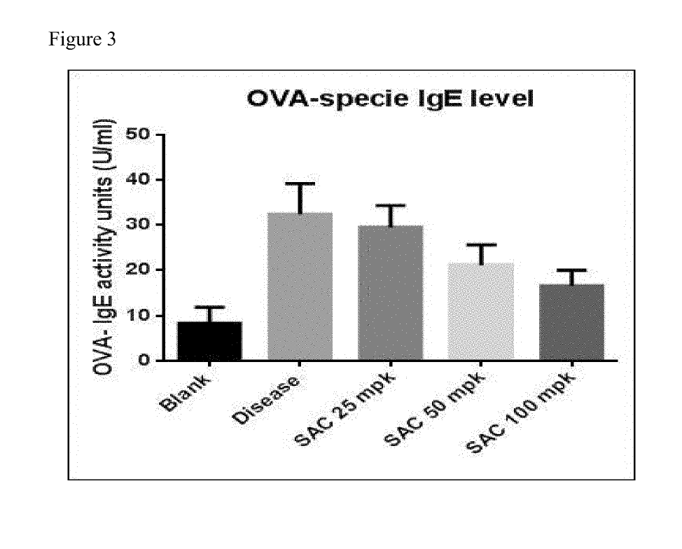

Changes of cytokines in nasal irrigation solution are shown in FIG. 3. In comparison with the disease group, the level of IL-6 and IL-9 decreased as the amount of SAC increased.

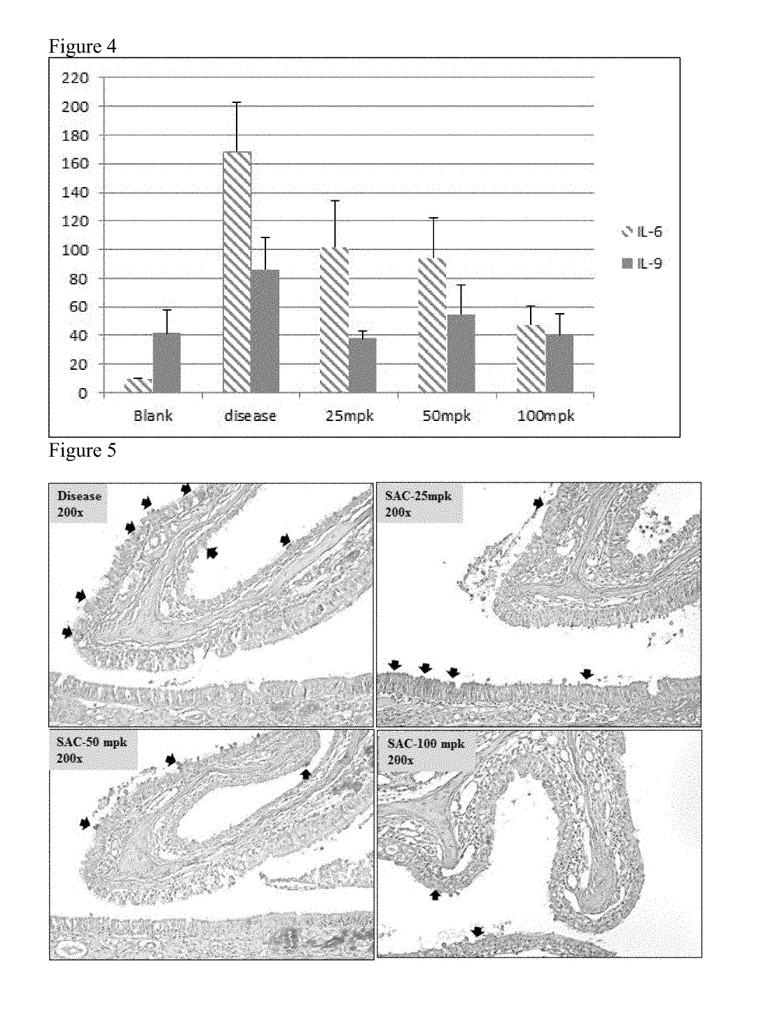

The results of the histopathological sections stained with +PAS staining of the nasal cavities are shown in FIG. 4.

From the above results it can be concluded that the composition of the present invention has the efficacy of prevention and treatment of allergic rhinitis.

1. A method for preventing or treating an immune allergic airway disease in a subject in need thereof, comprising administering a composition comprising an effective amount of S-allyl-L-cysteine and a pharmaceutically acceptable carrier.

2. The method of claim 1, wherein the immune allergic airway disease is indicated by immunoglobulin E expression level.

3. The method of claim 1, wherein the immune allergic airway disease is allergic rhinitis.

4. The method of claim 3, wherein the allergic rhinitis comprises symptoms of sneezing and nasal rubbing.

5. The method of claim 1, wherein the effective amount of S-allyl-L-cysteine for mice is at least about 25 mg/kg.

6. The method of claim 1, wherein the effective amount of S-allyl-L-cysteine for human is at least about 2.75 mg/kg.

7. The method of claim 1, wherein the pharmaceutically acceptable carrier comprises diluent, excipient, or acceptance agent.
